(19) United States
(12) Patent Application Publication
(10) Pub. No.: US 2009/0210280 A1
(43) Pub. Date: Aug. 20, 2009

(54) METHOD AND APPARATUS FOR CREATING A SCHEDULE BASED ON PHYSIOLOGICAL DATA

(75) Inventors: Sheng Jin, Shanghai (CN); Declan Patrick Kelly, Shanghai (CN)

Correspondence Address: PHILIPS INTELLECTUAL PROPERTY & STANDARDS, P.O. BOX 3001, BRIARCLIFF MANOR, NY, 10510, US

(73) Assignee: Koninklijke Philips Electronics N.V., Eindhoven (NL)

(21) Appl. No.: 12/305,463

(22) PCT Filed: Jun. 25, 2007
(86) PCT No.: PCT/IB2007/052441

§ 371 Date: Dec. 18, 2008

(30) Foreign Application Priority Data
Jun. 27, 2006 (CN) 200610100033.9

(51) Int. Cl. G06Q 10/00; A61B 5/00

(52) U.S. Cl. 705/9; 600/300

(57) ABSTRACT

The invention provides a method and apparatus for creating a schedule, wherein the schedule is used for a user's schedule arrangement, the apparatus for creating a schedule comprises: acquiring means for acquiring at least one physiological health condition data of the user; analyzing means for generating suggestion on the schedule arrangement based on said at least one physiological health condition data; and generating means for adding the schedule arrangement to the schedule according to a predetermined rule. The invention is used to dynamically create a schedule arrangement adaptive to the physiological health condition of a user under the premise that there is a change in the physiological health condition of the user, so that the scheduled exercises of the user are arranged more efficiently, securely and reasonably.

### FIELD OF THE INVENTION

The present invention relates to a method and apparatus for monitoring physiological health data, in particular to a method and apparatus for creating a schedule based on the monitored physiological health data.

### BACKGROUND OF THE INVENTION

In recently years, more and more technologies used for monitoring personal health care data have been set forth, moreover, many of these technologies are related to remote monitoring. For example, doctors in the hospital monitor the patients at home. As for such monitoring activity, a special monitoring device collects various personal health care data of the monitored patients and sends the health care data to the paramedic, for example, the product Motiva manufactured by Koninklijke Philips Electronics N.V., it combines many medical facilities, such as heart rhythm measurer, blood pressure measurer, and the instrument for measuring glucose. A patient suffering from heart disease may stay at home and measure basic functions of the physical body without going to the hospital for examination.

For old people, a schedule may help them overcome a bad memory and remind them of a schedule arrangement immediately. In addition, the health condition of old people is relatively instable, it is quite necessary to use a monitoring device to timely collect various personal health care data and send the health care data to the paramedic, a reasonable schedule is of great importance to old people. The healthy condition of old people will be affected if their daily exercises are not arranged reasonably, for example, it may result in an abnormal situation of the physiological parameters like blood pressure and rhythm of heart.

Usually, a relatively reasonable schedule is arranged by doctors according to the characteristics of the physical condition of old people. However, if the physiological health condition of an old man/woman has a change and becomes instable, the pre-arranged schedule is hard to reach the anticipate effect.

### OBJECT AND SUMMARY OF THE INVENTION

The present invention provides a method and arrangement for creating a schedule; it needs to provide a method and apparatus that can dynamically arrange a schedule based on the real-time physiological health condition of a user, and a method and apparatus that can arrange a schedule more reasonably based on the real-time physiological health condition of a user.

According to the method for creating a schedule as provided by the invention, the schedule is used for a user's schedule arrangement, the method comprises the steps of: acquiring at least one physiological health condition data of the user; generating suggestion on a schedule arrangement based on said at least one physiological health condition data; and adding the schedule arrangement to the schedule according to a predetermined rule.

According to the apparatus for creating a schedule as provided by the invention, the schedule is used for a user's schedule arrangement, said apparatus comprises: acquiring means for acquiring at least one physiological health condition data of the user; analyzing means for generating suggestion on a schedule arrangement based on said at least one physiological health condition data; and generating means for adding the schedule arrangement to the schedule according to a predetermined rule.

The computer program product for creating a schedule as provided according to by the invention comprises: code for acquiring at least one physiological health condition data of the user; code for generating suggestion on a schedule arrangement based on said at least one physiological health condition data; and code for adding the schedule arrangement to the schedule according to a predetermined rule.

In summary, the method and apparatus for creating a schedule as provided by the invention can dynamically create a schedule arrangement adaptive to the physiological health condition of a user under the premise that there is a change in the physiological health condition of the user, so that the scheduled exercises of the user are arranged more efficiently, securely and reasonably.

Other objects and achievements of the invention will become obvious through the following description with reference to the drawings and the claims, and a comprehensive understanding of the invention will be obtained.

### BRIEF DESCRIPTION OF THE DRAWINGS

FIG. 1 is a schematic diagram of the apparatus for creating a dynamic schedule according to an embodiment of the invention.

FIG. 2 is a flow chart illustrating the method for creating a dynamic schedule according to another embodiment of the invention.

FIG. 3 is a schematic diagram of a display interface of the apparatus for creating a dynamic schedule according to an embodiment of the invention.

In all drawings, the same sign represents the same, similar or corresponding feature or function.

### DETAILED DESCRIPTION OF THE INVENTION

Below the preferable embodiments of the invention are described in detail in combination with the drawings.

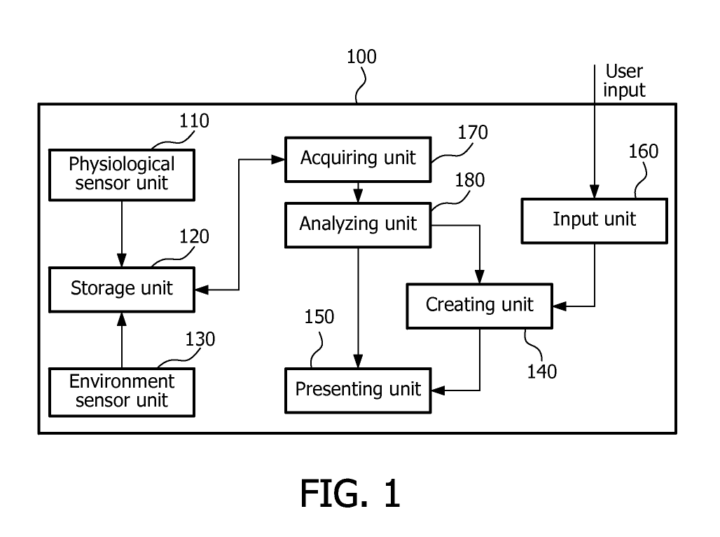

FIG. 1 is a schematic diagram of the apparatus for creating a dynamic schedule according to an embodiment of the invention.

The schedule creating apparatus 100 comprises an acquiring unit 170, an analyzing unit 180 and a creating unit 140.

The schedule creating apparatus 100 further comprises a storage unit 120 for storing data, i.e. the monitored user physiological health data and environment data. The storage unit 120 may be a non-volatile flash card, or any other storage means, for example, a hard disk, a floppy disk, etc.

According to an embodiment of the invention, the storage means 120 is further used to store a database of physiological health data. The database includes the normal range of each item of physiological health data and the corresponding suggestion where each item of physiological health data is inconsistent with the normal data range, for example, if the value of blood pressure is higher than a normal range, the user is advised to take the exercises like relax and rest instead of making strenuous exercises. The database of physiological health data may be established by the professional doctors or the patient's supervisors according to the user's healthy condition and medical professional knowledge.

According to another embodiment of the invention, the database may include the range of data of each physiological health index under various different situations, instead of merely comprising the normal range and abnormal range of one item of physiological health data. For example, for the blood oxygen concentration of SPO₂, it may set a plurality of data ranges: 70%-80%, 80%-90% and 90%-100%. According to the range of data in which the user's physiological index is included, different suggestion on schedule arrangement is created correspondingly, such as having a rest and going to see the doctor.

Table 1 shows an example of the database of physiological health data and is an energy consumption contrast table of various types of exercises, the schedule creating apparatus 100 may produce corresponding suggestion on activity arrangement based on the amount of the energy consumption of the user's exercises.

TABLE 1

| | Energy consumption of |
|---|---|
| Types of exercises | exercises (Kcal/min) |
| Sleep or rest | 1.0 |
| Sit down quietly | 1.4 |
| Walk (5 km per hour, unloaded) | 3.7 |
| Walk (5 km per hour, 10 kg loaded) | 4.0 |
| Light exercises (billiards, golf, etc.) | 2.5-5.0 |
| Moderate exercises (boating, dancing, | 5.0-7.5 |
| swimming, etc.) | |
| Heavy exercises (football, etc.) | Above 7.5 |

The acquiring means 170 is used to acquire from the storage means 120 the real-time physiological health data of the user and the corresponding database of the physiological health data.

According to an embodiment of the invention, the schedule creating apparatus 100 further comprises an analyzing unit 180 for comparing the user real-time physiological health data acquired by the acquiring unit 170 with the corresponding database of physiological health data and analyzing it to thereby obtain corresponding suggestion on schedule arrangement.

The analyzing unit 180 is used to analyze the physiological health condition data acquired by the acquiring unit 170 to obtain a piece of suggestion on a schedule arrangement, the created suggestion on schedule arrangement is adaptive to the acquired physiological health condition data. For example, if the physiological health condition data acquired by the acquiring unit 170 indicates that the pulse value of the user is high, the analyzing unit 170 may analyze and generate a piece of schedule arrangement suggestion that suggests the user having a rest, said schedule arrangement suggestion is created based on the database of the physiological health data stored in the aforesaid storage unit 120.

According to an embodiment of the invention, the schedule creating apparatus 100 further comprises a creating unit 140 for adding, based on a predetermined rule, the schedule arrangement suggestion analyzed and obtained by the analyzing unit 180 to the user's schedule. Said predetermined rule may be the information input by the user for confirming whether to accept the schedule arrangement suggestion, or the information set according to the personal physiological health characteristics of the user.

For example, the analyzing unit 180 will, each time when it analyzes and obtains a piece of schedule arrangement suggestion, present the suggestion to the user via a presenting unit 150, so as to give the user a prompt about whether to accept this piece of suggestion. Alternatively, the user may set a rule in advance according to his/her personal physiological health characteristics to automatically give a response to whether to accept the schedule arrangement suggestion. For example, the user may automatically accept all schedule arrangement suggestion relating to the data of the blood oxygen concentration of SPO₂ and blood pressure, whereas the schedule arrangement suggestion related to other physiological health data are presented to the user via the presenting unit 150 so as to give the user a prompt about whether to accept such piece of suggestion.

According to an embodiment of the invention, the schedule creating apparatus 100 further comprises a sensor unit 110. The sensor unit 110 is used to monitor the physiological health condition of a user, and includes a blood pressure biological sensor for detecting the blood pressure data of the user. The sensor unit 110 may further include other types of biological sensors used for monitoring other physiological data of the user, for example, body temperature, heart rate, the blood oxygen concentration of SPO₂, etc.

According to an embodiment of the invention, the physiological sensor unit 110 may further comprise an activity detection sensor for monitoring the user's exercises, for example, an accelerator may be applied to the acceleration speed of the user's exercises.

According to an embodiment of the invention, the schedule creating apparatus 100 further comprise an environment sensor unit 130 for monitoring the environment data associated with the environment in which the user is situated, for example, the indoor temperature, humidity, noise level, geographical location positioning, etc. The analyzing unit 180 may be used to analyze the physiological health condition data acquired by the acquiring unit 170 and combine the environment data associated with the environment in which the user is situated to obtain a pieced of suggestion on the schedule arrangement.

According to another embodiment of the invention, the physiological sensor unit 110 and environment sensor unit 130 may be independent of the schedule creating apparatus 100, as long as the sensor unit 110 and activity detection sensor unit 130 can establish a communication channel and realize data transmission with the receiving means 100 by means of any current or future wire/wireless communication measures, for example, the data is transmitted in a Blue-tooth protocol.

According to an embodiment of the invention, the schedule creating apparatus 100 further comprises a presenting unit 150. The presenting unit 150 is used to present the schedule arrangement suggestion created by the analyzing unit 180, and the schedule created by the creating unit 140. In this embodiment, a display presents the physiological health data/environment data and schedule arrangement suggestion/schedule. For example, different font colors are used with regard to different types of data, green is used for the blood pressure, yellow is used for the body temperature, weight and noise level, red is used for any data beyond the normal range of data. Another example is different background colors are used for different types of data.

According to another embodiment of the invention, it may also use any other presenting apparatus such as a loudspeaker to assist in presenting the data.

According to an embodiment of the invention, the schedule creating apparatus 100 further comprises an input unit 160 for receiving the user's input to confirm whether to accept the created schedule arrangement suggestion. Alternatively, the input unit receives the user's inputs and amends the created schedule arrangement suggestion.

It shall be understood that according to an embodiment of the invention, part of the function of the schedule creating apparatus 100 shown in FIG. 1 can also be realized by means of an appropriately programmed computer, the computer is installed with a computer program for creating a schedule. The computer program includes: a code for acquiring at least one physiological health condition data of the user; a code for generating suggestion on a schedule arrangement based on said at least one physiological health condition data; and a code for adding the schedule arrangement to the schedule according to a predetermined rule.

The computer program product as mentioned above may be stored in a storage carrier.

These parts of the aforesaid computer program codes are provided to a processor for generating a machine so that the codes executed on the processor create means for realizing the aforesaid function.

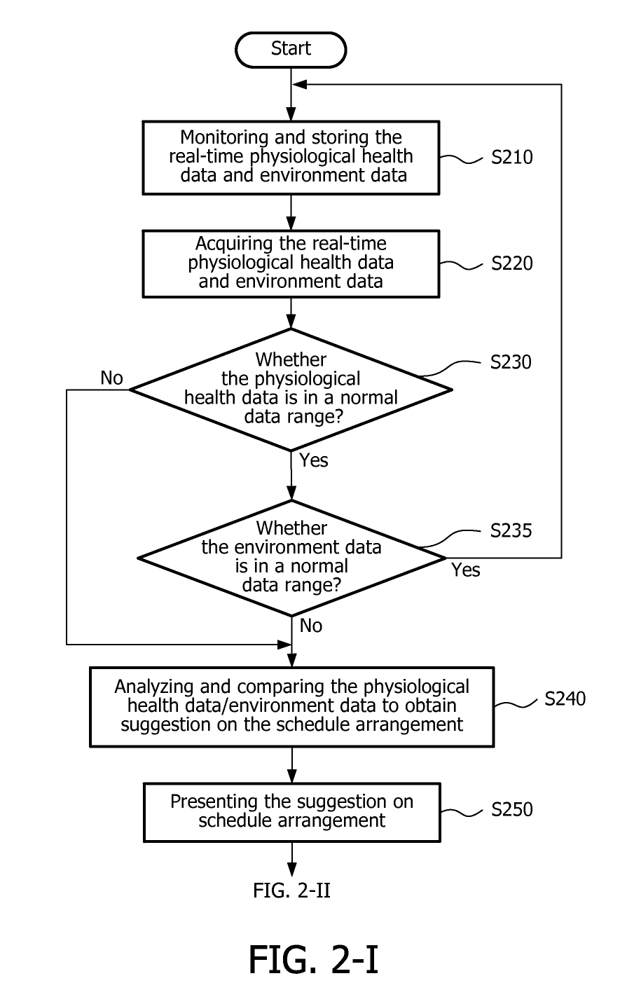

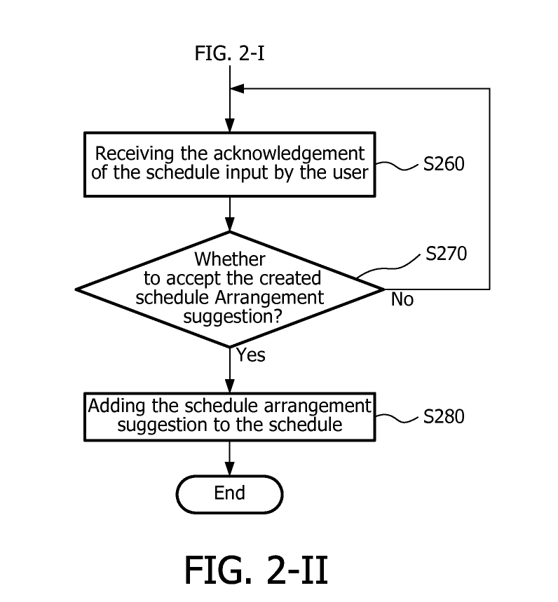

FIG. 2 is a flow chart illustrating the method for creating a dynamic schedule according to an embodiment of the invention. The method may be applied to the schedule creating apparatus 100 as shown in FIG. 1.

First of all, an initial schedule is created according to the user's initialized setting, the initial schedule may be established by the professional doctors or the patient's supervisors, the user's initialized setting information contain the settings such as the user's current physiological health information and the user's living habits. For example, the user's blood pressure is high, so the user is not suitable for strenuous exercises; and the relevant setting information of the personal preference, such as the user likes walking in the afternoon. The initialized schedule will, first of all, consider the user's physiological health information to arrange the schedule and exercises, and then consider the user's personal living habit information to arrange schedule and exercises.

In step S210, monitoring and storing the user's real-time physiological health data and environment data. Physiological data may include, for example, body temperature, heart rhythm, SPO₂ blood oxygen concentration, and etc., the environmental data associated with the environment may include, for example, the indoor temperature, humidity, noise level, and etc.

In step S220, acquiring the aforesaid stored user's real-time physiological health data/environment data and corresponding database of the physiological health data.

In step S230, judging whether the monitored real-time physiological health data is in a normal range of data. It may compare the real-time physiological health data with a physiological health database, for example, the monitored real-time blood pressure value is 168 mmHg-110 mmHg, whereas the normal range of blood pressure is 134 mmHg-82 mmHg, this explains the value of the user's blood pressure is on the high side and is not in a normal range.

According to another embodiment of the invention, the physiological health database may comprise the range of data of each physiological health index under various different situations, instead of merely comprising the normal range and abnormal range of one item of physiological health data. For example, for the blood oxygen concentration of SPO₂, it may set a plurality of data ranges: 70%-80%, 80%-90% and 90%-100%. According to the ranges of data in which the user's physiological index is included, different suggestion on schedule arrangement is created correspondingly, such as having a rest, and going to see the doctor.

If, after judgment, the monitored real-time physiological health data is out of a normal data range, the schedule creating apparatus will remind the user of adding certain schedule arrangement relating to rest to the schedule.

According to another embodiment of the invention, the varying tendency of some physiological health data may be used as the basis of modifying the schedule. For example, within a certain period of time, it is found that the pulse frequency of the user keeps rising, then the schedule creating apparatus 100 will remind the user and suggest the user adding certain time for rest to the schedule.

If the monitored real-time physiological health data is in a normal data range, then in step S235, it is judged whether the monitored real-time environment data is in a normal data range.

If the environment data is not in a normal data range, in step 240, it compares the acquired user's real-time physiological health data with the corresponding database of the physiological health data and analyzes it to obtain a corresponding suggestion on the schedule arrangement. The created schedule arrangement suggestion is adaptive to the received physiological health condition data. For example, if the received physiological health condition data indicates that the pulse value of the user is on the high side, it may generate a piece of schedule arrangement suggestion that suggest the user having a rest, the schedule arrangement suggest may be created based on the stored database of the physiological health data.

In step S250, presenting to the user a piece of schedule arrangement suggestion so as to obtain the user's acknowledgement. In this embodiment, the schedule arrangement suggestion is presented by a display.

In step S260, receiving the user's input to confirm the created schedule arrangement, or receiving the user's input to modify the created schedule arrangement.

In step 270, judging whether to confirm the created schedule arrangement. If so, in step 280, adding the suggestion on said schedule arrangement to the user's schedule according to the user's acknowledgement information. According to another embodiment of the invention, if the created schedule arrangement suggestion is in conflict with the user's initial schedule, said created schedule arrangement replaces the schedule arrangement in the initial schedule. Finally, ending the whole flow procedure.

If not, returning to step S260 again, and continuing to receive the user's input.

According to another embodiment of the invention, it may determine, based on a predetermined rule, whether to add the schedule arrangement suggestion to the user's schedule, wherein said predetermined rule may include the acknowledgment information input by the user about whether to accept the schedule arrangement suggestion, or the acknowledgement information set according to the user's personal physiological health characteristics.

For example, acquiring each piece of suggestion on schedule arrangement and presenting it to the user so as to give the user a prompt of whether to accept this suggestion. Alternatively, the user may set a rule in advance based on his/her personal physiological health characteristics to automatically make a response on whether to accept the suggestion on schedule arrangement. For example, the user may automatically accept all schedule arrangement suggestion relating to the blood oxygen concentration of SPO₂ and the blood pressure data, whereas the schedule arrangement suggestion relating to other physiological health data are presented to the user, thereby giving the user a prompt of whether to accept this suggestion.

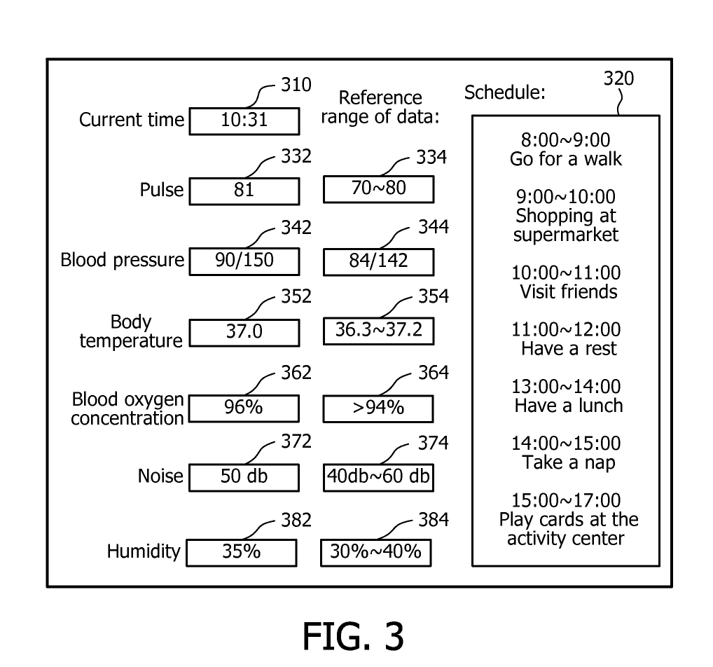

FIG. 3 is a schematic diagram of a display interface of the apparatus for creating a dynamic schedule according to an embodiment of the invention.

Domain 310 represents the data monitored current time.

Domain 320 represents the schedule arrangement of the user at the current time (the current day).

Domain 332 displays “82” in red, indicating the user's current pulse is 82 times per minute, this means the current pulse is higher than a normal data range of the pulse. Domain 334 displays “70-80”, indicating the normal data range of the times of pulse.

Domain 342 displays “90/150” in red, indicating the current value of the user's blood pressure is 90/150, this means the current blood pressure value is on a high side of the normal data range of the blood pressure value. Domain 344 displays “84/142”, indicating the normal data range of the blood pressure value.

Domain 352 displays “37.0”, indicating the value of user's body temperature is 37.0 degree, and domain 354 indicates the paramedic may initiate a request for the instant data of the temperature.

Domain 362 displays “96%”, indicating the user's blood oxygen concentration of SPO₂ is 96%, domain 364 displays “>% 94”, indicating this blood oxygen concentration of SPO₂ is in a normal data range.

Domain 372 displays “50 db”, indicating the noise of the environment in which the user is situated is 50 db. Domain 374 displays “40 db-60 db”, indicating this environment noise is in a normal data range.

Domain 382 displays “35%”, indicating the relative humidity of the environment in which the user is situated is 35%. Domain 364 displays “30-40%”, indicating this environment humidity is in a normal data range.

According to an embodiment of the invention, the aforesaid domains 334, 344, 354 and 364 indicating a normal range may be used to display a plurality of different data ranges of the physiological health index data, the plurality of different data ranges may correspond to different health and safety levels, respectively, for example, a plurality of safety levels such as normal, caution, dangerous, etc.

According to an embodiment of the invention, the schedule arrangement displayed by domain 320 may be adjusted in accordance with the user's real-time physiological health data. For example, as shown in FIG. 3, at the current time 9:53, it can be known after monitoring that the user's pulse value is 82 and the blood pressure value is 90/150, both are higher than the normal data range, however, according to the schedule arrangement at the current time, the user plans to visit friends between 10:00-11:00. Now, the schedule creating apparatus may create a new schedule arrangement conforming to the current physiological index in accordance with the user's physiological index, for example, to have a rest or go for a walk, as a replacement of the original schedule arrangement that is not suitable for the current physiological index.

According to another embodiment of the invention, the schedule creating apparatus may create a new schedule arrangement in accordance with the user's physiological index and recommend it to the user, and after the user inputs confirmation, the apparatus replaces the original schedule arrangement with the newly created schedule arrangement.

Those skilled in the art shall understand that the method and apparatus for creating a schedule as disclosed by the invention may have various improvements on the basis of not breaking away from the contents of the present invention. Therefore, the protection scope of the invention shall be defined by the contents of the appended Claims.

1. A method for creating a schedule, said schedule being used for a user's schedule arrangement, said method comprises the steps of:
(a) acquiring at least one physiological health condition data of the user;
(b) generating suggestion on the schedule arrangement based on said at least one physiological health condition data; and
(c) adding the schedule arrangement to the schedule according to a predetermined rule.

2. The method according to claim 1, wherein said predetermined rule is an acknowledge information input by the user.

3. The method according to claim 1, wherein said predetermined rule is an acknowledgement information that is set according to the user's personal physiological health characteristics.

4. The method according to claim 1, further comprising the step of:
monitoring at least one physiological condition data of the user;
wherein step (a) includes the step of acquiring the at least one monitored physiological health condition data.

5. The method according to claim 1, further comprising the step of:
acquiring at least one environment condition data of the user;
wherein step (b) includes the step of generating suggestion on schedule arrangement according to the at least one physiological health condition data and the environment condition data.

6. The method according to claim 1, wherein step (b) includes the steps of: judging whether said physiological condition data is within a reference data range; and generating suggestion on schedule arrangement based on the result of judging.

7. The method according to claim 1, further comprising the step of:
presenting said suggestion on said schedule arrangement to the user.

8. An apparatus for creating a schedule, said schedule being used for a user's schedule arrangement, said apparatus comprising:
(a) acquiring means for acquiring at least one physiological health condition data of the user;
(b) analyzing means for generating suggestion on the schedule arrangement based on said at least one physiological health condition data; and
(c) generating means for adding the schedule arrangement to the schedule according to a predetermined rule.

9. The apparatus according to claim 8, wherein said predetermined rule is an acknowledge information input by the user.

10. The apparatus according to claim 8, wherein said predetermined rule is an acknowledgement information that is set according to the user's personal physiological health characteristics.

11. The apparatus according to claim 8, further comprising:
a physiological sensor means for monitoring at least one physiological condition data of the user;
wherein said acquiring means is used to acquire the at least one monitored physiological health condition data.

12. The apparatus according to claim 8, wherein said acquiring means is further used to acquire the user's at least one environment condition data; wherein said analyzing means is used to generate suggestion on schedule arrangement according to said at least one physiological health condition data and environment condition data.

13. The apparatus according to claim 8, further comprising:
a presenting means for presenting said suggestion on said schedule arrangement to the user.

14. A computer program product for creating a schedule, comprising: code for acquiring at least one physiological health condition data of the user;
code for generating suggestion on a schedule arrangement based on said at least one physiological health condition data; and
code for adding the schedule arrangement to the schedule according to a predetermined rule.
